(19) United States
(12) Patent Application Publication
(10) Pub. No.: US 2014/0052162 A1
(43) Pub. Date: Feb. 20, 2014

(54) MEDICAL DEVICE HAVING A LATTICE STRUCTURE AND TREATMENT SYSTEM HAVING SUCH A LATTICE STRUCTURE

(71) Applicant: Giorgio Cattaneo, Karlsruhe (DE)

(72) Inventor: Giorgio Cattaneo, Karlsruhe (DE)

(73) Assignee: ACANDIS GMBH & CO. KG, Pfinztal (DE)

(21) Appl. No.: 13/981,346

(22) PCT Filed: Jan. 25, 2012
(86) PCT No.: PCT/EP2012/051118

§ 371 Date: Oct. 7, 2013

(30) Foreign Application Priority Data
Jan. 25, 2011 (DE) 10 2011 009 372.9

(51) Int. Cl. A61B 17/221; A61M 25/00

(52) U.S. Cl. CPC A61B 17/221; A61M 25/0082
USPC 606/159

(57) ABSTRACT

Medical device having compressible and expandable, circular cylindrical lattice structure, which includes circumferential segments having closed cells. The cells are bounded by four respective webs, which are coupled to each other and every two oppositely arranged webs to form a web pair. The webs of a first pair have a different shape and/or a web width at least in some sections than the webs of a second pair. The webs of the first pair can be deformed more greatly than the webs of the second pair. During the transition of the lattice structure from the expanded to the compressed state, each web of the first pair is coupled to a respective web of the second pair in such a way that two connection points oppositely arranged in the longitudinal direction (LR) of the lattice structures are moved away from each other in the circumferential direction (UR) of the lattice structure.

The invention relates to a medical device with a compressible and expandable, circular cylindrical lattice structure comprising circumferential segments made of closed cells.

The technical field of the invention comprises, in particular, stent-like systems and devices for treating diseases of the cardiovascular system. It includes, for example, devices for removing blood clots, in particular thrombectomy devices.

Practice has disclosed thrombectomy devices, which comprise a basket-like lattice structure. The lattice structure can be expanded and compressed in the radial direction and is brought to the treatment location by a supply catheter. Here, the lattice structure is available in the compressed state in the supply catheter. By the release from the supply catheter at the treatment location, the basket-like lattice structure widens or expands. As a result of the expansion, the lattice structure has a larger cross-sectional diameter within a blood vessel than within the supply catheter, i.e. in the compressed state.

The lattice structure is formed by webs which delimit cells. During the transition of the lattice structure from the radially compressed state into the radially expanded state, the webs of the lattice structure move only in the radial direction. Proceeding from a longitudinal axis of the lattice structure, the webs of the lattice structure move apart only in the radial direction. Conversely, there is a radial movement of the webs in the direction of the longitudinal axis during the compression of the lattice structure.

At the treatment location, i.e. for example in the region of a thrombus, the lattice structure of known devices is expanded such that the webs cut into the thrombus or the blood clot in a substantially straight-lined radial direction. As a result, the lattice structure and the blood clot are connected.

In order to remove the blood clot connected to the lattice structure from the blood vessel, the lattice structure or, in general, the known device is pulled out of the blood vessel, with the lattice structure also passing through blood vessels that have a larger cross-sectional diameter than the treatment location. In the process, there is the risk that the blood clot, which is substantially connected to the webs of the lattice structure by friction, is detached in the radial direction and carried along by the blood flow in the vessel. As a result, the blood clot or at least parts of the blood clot can lead to a new closure of a blood vessel.

Known devices are also employed to detach blood clots more easily from the vessel wall. To this end, the expanded lattice structure is, e.g. from a proximal end of the blood clot, pushed into the blood clot along the vessel wall, with the lattice structure simultaneously being rotated by hand. This means that the user attempts to bring about a rotation of the lattice structure arranged at the distal end of a guide wire by a rotation at the proximal end of the guide wire. In practice, this is found to be particularly difficult since, firstly, the circumferential area of a thin guide wire is too small to be able to apply sufficient frictional forces between the fingers of the user and the guide wire and rotate the guide wire. Furthermore, a rotation of the proximal end of the guide wire initially brings about a twist in the fine guide wire, and so a rotation of the lattice structure arranged at the distal end of the guide wire either does not occur at all or only occurs with a great time delay. It is therefore difficult to control the rotation of the lattice structure in the known devices.

The object of the invention consists of specifying a medical device with a compressible and expandable, circular cylindrical lattice structure, which enables improved anchoring in a blood clot, is simple to control and is easy to handle. Furthermore, the object of the invention lies in specifying a treatment system with such a device.

According to the invention, this object is achieved by the subject matter of patent claim 1 in respect of the medical device and by the subject matter of patent claim 8 in respect of the treatment system.

The invention is based on the idea of specifying a medical device with a compressible and expandable, circular cylindrical lattice structure comprising circumferential segments made of closed cells. The cells are each delimited by four webs which are coupled to one another at connection sites and of which two webs, respectively arranged opposite to one another, have the same design and form a web pair. The webs of a first web pair have, at least in sections, a different shape and/or a different web width than the webs of a second web pair in such a way that the webs of the first web pair are deformed more during the transition of the lattice structure from the expanded state into the compressed state than the webs of the second web pair. Respectively one web of the first web pair is coupled to one web of the second web pair in such a way that two connection sites arranged opposite to one another in the longitudinal direction of the lattice structure are offset in the opposite direction in the circumferential direction of the lattice structure during the transition of the lattice structure from the expanded state into the compressed state. All cells of a circumferential segment have the same design such that the whole lattice structure twists, at least in sections, during the transition from the expanded state into the compressed state.

In accordance with the coordinate aspect, the invention is based on the idea of specifying a treatment system with the medical device and a catheter, wherein the medical device comprises a guide element, more particularly a guide wire, which is fixedly, more particularly rotationally fixedly, connected to an axial end of the lattice structure and arranged in a longitudinally displaceable fashion in the catheter.

The individual webs of the lattice structure are deformed the transition of the lattice structure from the expanded state into the compressed state, i.e. during the compression of the lattice structure. This deformation is undone during the expansion of the lattice structure, i.e. during the transition of the lattice structure from the compressed state into the expanded state. Alternatively, provision can be made for the deformation of the webs to take place during the expansion and for the webs to stretch again during the compression. In particular, the individual webs are deformed in an elastic range. The expansion or compression of the lattice structure relates to the cross-sectional diameter. During the compression, the cross-sectional diameter of the lattice structure reduces, whereas the cross-sectional diameter increases during the expansion.

In the invention, provision is made for respectively two webs of a cell, arranged opposite to one another, to form a web pair, the webs of which are deformed differently in the case of the state change of the lattice structure, i.e. during the compression and/or during the expansion, than the webs of a further web pair of the same cell. In particular, the webs of a web pair have the same properties. Specifically, provision is made for the webs of a first web pair to have a different shape and/or, at least in sections, a different web width than the webs of a second web pair. Here, a web pair is formed by respectively two webs, which are arranged lying opposite to one another in the cell, i.e. which are not coupled to one another at a connection site. It is rather the case that two webs from different web pairs are respectively coupled to one another at the connection sites.

What the different shape and/or web width of the webs of the first web pair with respect to the webs of the second web pair, at least in sections, achieves is that the connection sites, arranged lying opposite to one another in the longitudinal direction, of a cell are offset in the opposite direction in the case of the state change of the lattice structure, i.e., for example, during the transition from the compressed state into the expanded state. In other words, during the state change of the lattice structure, there is not only a movement of the connection sites, arranged opposite to one another in the longitudinal direction, parallel to the longitudinal axis of the lattice structure, i.e. in the longitudinal direction, but also a movement in the circumferential direction.

The lattice structure comprises a plurality of circumferential segments, which each comprise cells with the same design. The circumferential segments have a substantially annular design and comprise closed cells, i.e. cells that are delimited by webs on all sides. What the aforementioned differences in shape and/or web width between the webs of the first web pair and those of the second web pair brings about is that the cells of a circumferential segment rotate substantially about a rotational point arranged in the cell, as a result of which, overall, a twist is set, at least in sections, along the lattice structure. This means that, during the transition from the compressed state into the expanded state, and vice versa, the webs of the lattice structure move not only in a straight line in the radial direction, but at the same time carry out a movement in the circumferential direction. During the expansion of the lattice structure in the region of a blood clot or thrombus, what this brings about is that the webs cut into the blood clot or the thrombus not only in a straight line, but substantially in a screw-like fashion. The anchoring of the lattice structure or, in general, the medical device in a concretion, in particular a blood clot or thrombus, is thus improved.

The twist of the lattice structure is already brought about by the expansion per se. The expansion preferably occurs automatically as soon as an external force applied by a supply catheter is removed. In other words, the lattice structure preferably expands automatically when it is released from the supply catheter. The release from the supply catheter is brought about by a longitudinally axial relative motion between the supply catheter and the lattice structure. Hence the twist of the lattice structure and the circumferential movement of the webs of the lattice structure are already achieved by a translational relative movement between the supply catheter and the lattice structure. This significantly improves the handling of the medical device. In particular, the twist of the lattice structure is easy to control since a translational relative movement can be transmitted better over a guide wire than a rotational movement.

In accordance with a preferred embodiment of the device according to the invention, each cell comprises four connection sites, which span a diamond-shaped basic shape of the cell in the expanded state of the lattice structure. This applies, in particular, to the completely expanded state, i.e. the production state of the lattice structure. Here, provision is made for the imagined connecting straight lines between the connection sites of a cell, connected by webs, together to form a diamond shape or, in general, a parallelogram shape. There preferably is a diamond shape of the cell at least in one intermediate state between the completely compressed state and the completely expanded state of the lattice structure. As a result of the state change of the lattice structure and the offset in opposite directions connected therewith, of the connection sites arranged opposite to one another in the longitudinal direction, the individual cell deforms in such a way that the basic shape merges into a parallelogram-like shape. The diamond-shaped basic shape of the cell is particularly advantageous for the twist in the lattice structure.

In a further embodiment of the medical device according to the invention, provision is made for the webs of the first web pair to have a substantially S-shaped embodiment and the webs of the second web pair to have a substantially straight embodiment. The webs deformed in an S-shaped manner deform more strongly during the state change of the lattice structure than the webs with a straight embodiment.

Alternatively, or in addition thereto, the webs of the first web pair can have a web width which is less than the web width of the webs of the second web pair. Thus, the first web pair can differ from the second web pair by the different width of the webs. Here, the webs of a single web pair have the same design, i.e. they have the same web width. It is also possible for the web width to differ only in sections along the webs. This means that the first web pair can have webs which each comprise a section in which the web width of the webs of the first web pair is smaller than the web width of the webs of the second web pair. The webs of the first web pair which, at least in sections, have a smaller web width than the webs of the second web pair therefore have a comparatively higher deformability during the state change of the lattice structure.

The different shape and/or the different web width between the webs of the first web pair and the webs of the second web pair can, in a further preferred embodiment, be represented by bending sites, which are arranged in the webs of the first web pair. The webs of the first web pair can thus each have a bending site, at which the web width and/or the web thickness of the respective web is reduced or increased, at least in sections. Alternatively, or in addition thereto, the bending site can also be achieved by a change in shape of the respective web. By way of example, the web can have at least one perforation and/or window, which forms a bending site. Overall, the overall width and/or the overall cross-sectional area of the respective web is reduced in the region of the bending site, and so, substantially, intended bending sites or kink points are formed, at which the web can be deformed comparatively easily. Thus, as a result of the bending sites, the webs of the first web pair are more deformable than the webs of the second web pair.

What the aforementioned design features bring about, either together or on their own, is that the webs of the first web pair are more deformable than the webs of the second web pair during the state change of the lattice structure, i.e. during the transition from the expanded state into the compressed state and vice versa. Here, the deformability of the webs of an individual web pair amongst themselves is the same. What this brings about is that an equal offset between the connection sites arranged opposite to one another in the longitudinal direction sets in during the state change of the lattice structure. In other words, proceeding from a zero-point position, the connection sites arranged opposite to one another in the longitudinal direction are moved by the same absolute value in the circumferential direction of the lattice structure, with the movement of the two connection sites being in opposite directions. Thus, a first connection site of the cell moves in the clockwise direction along the circumference of the lattice structure by the same value that the connection site, arranged opposite thereto in the longitudinal direction, moves in the counterclockwise direction.

The webs of a cell are integrally connected at the connection sites. This is how a relative movement of the webs amongst themselves is prevented. The deformation of the webs during the transition from the compressed state of the lattice structure into the expanded state and vice versa is therefore achieved by flexible or elastic bending or deflection of the individual webs. This achieves particularly high stability of the overall lattice structure.

In a further preferred embodiment of the medical device according to the invention, the circumferential segments each comprise two partial segments, which each have webs arranged in a meandering fashion, wherein every second web of a partial segment has the same design. The partial segments are therefore formed by webs arranged in a meandering fashion, wherein the webs are respectively coupled to one another at connection sites. Every second web of the partial segment has the same shape and/or web width. In other words, differently deformable webs are arranged alternately in a meandering fashion in a partial segment. The production of the device is therefore made simpler.

The invention will be explained in more detail below on the basis of exemplary embodiments, with reference being made to the attached schematic drawings. In detail

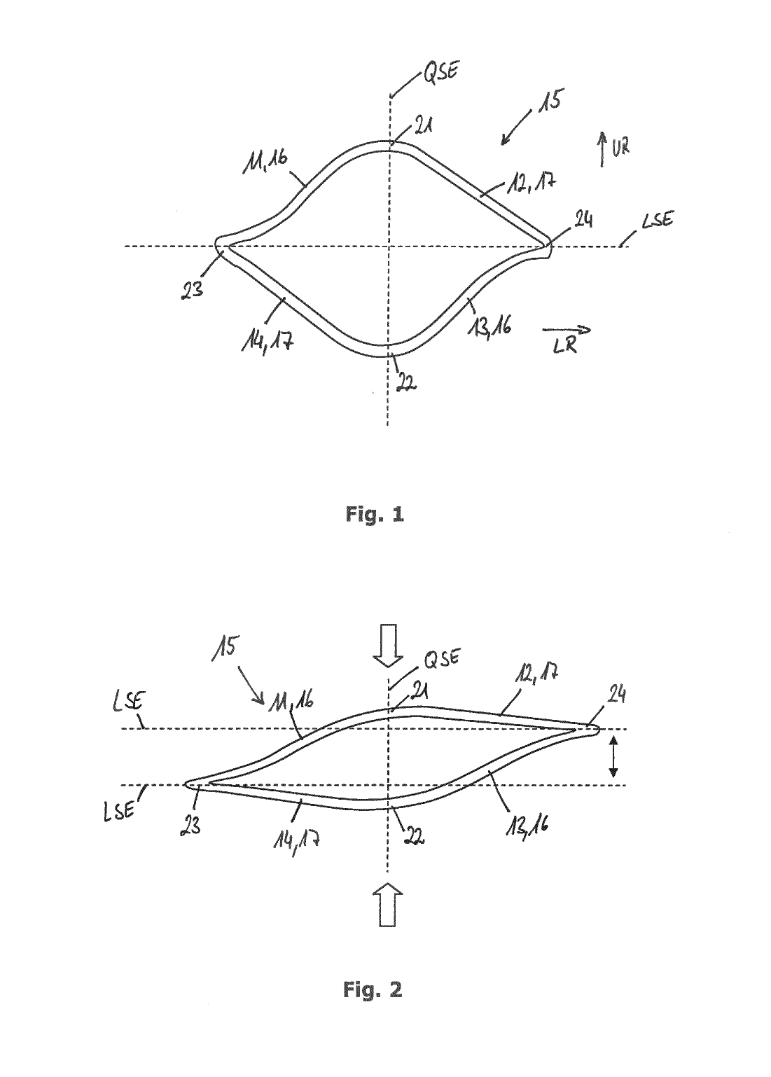

FIG. 1: shows a top view of a cell of the lattice structure of the medical device according to the invention according to a preferred exemplary embodiment, in the expanded state;

FIG. 2: shows the cell as per FIG. 1, in the compressed state;

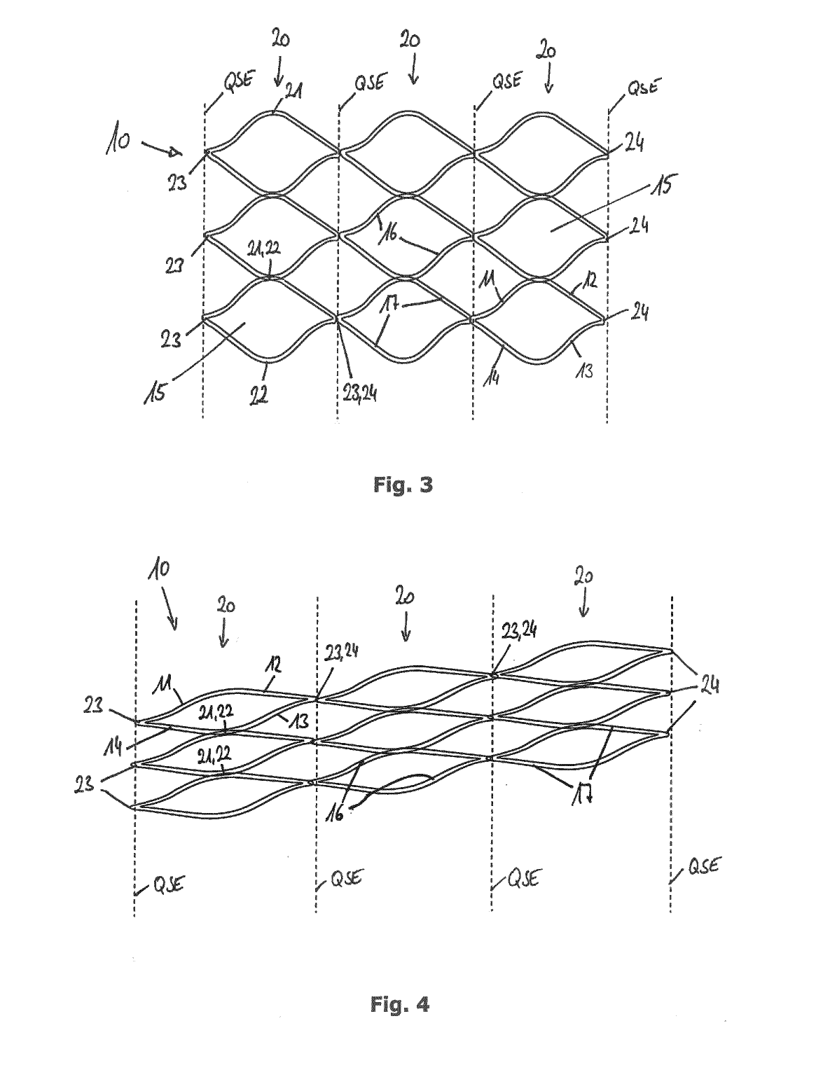

FIG. 3: shows a section of the lattice structure with several cells as per FIG. 1, in the expanded state;

FIG. 4: shows the lattice structure as per FIG. 3, in the compressed state;

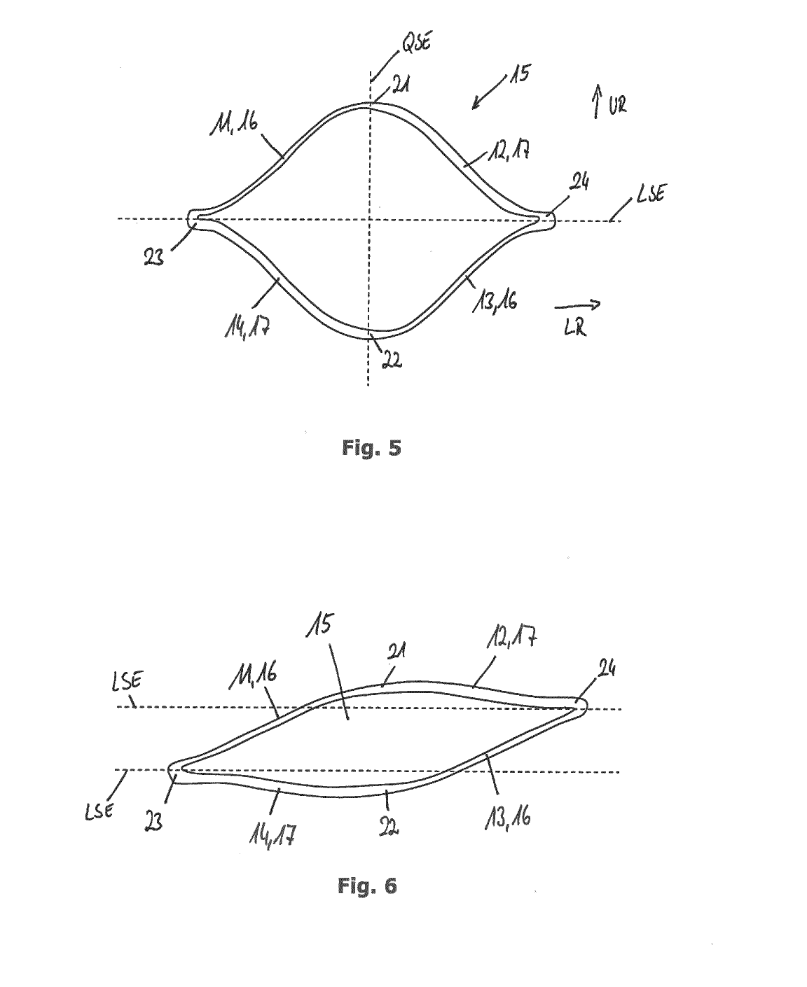

FIG. 5: shows a top view of a cell of the lattice structure of the medical device according to the invention according to a further preferred exemplary embodiment, in the expanded state, wherein the webs of the web pairs differ in terms of their web width;

FIG. 6: shows the cell as per FIG. 5, in the compressed state;

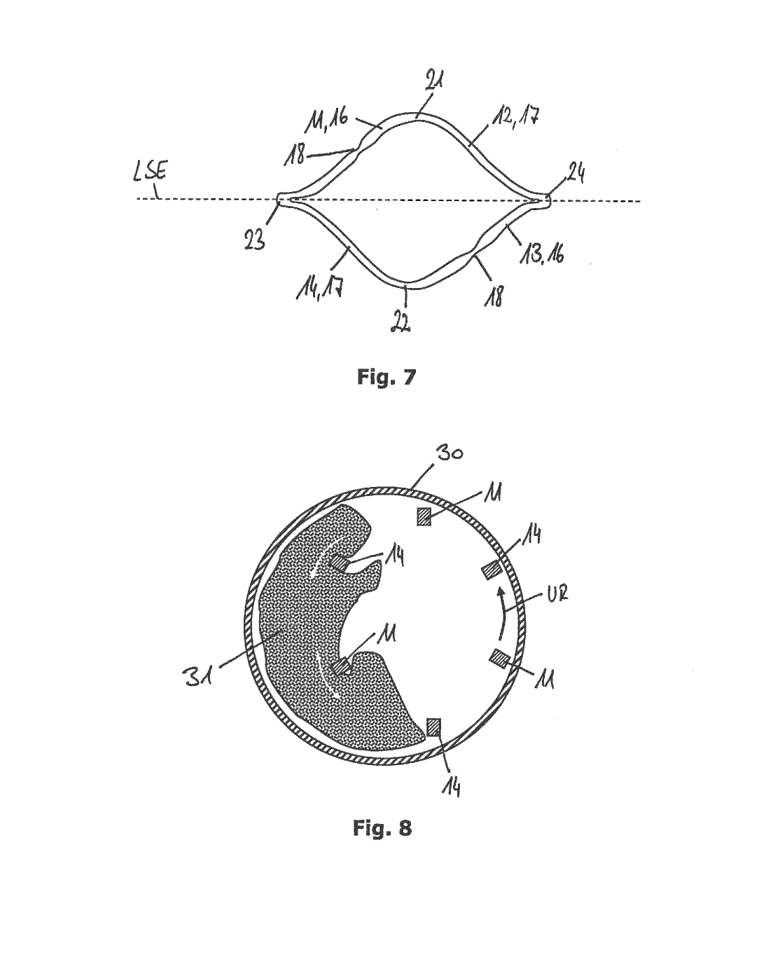

FIG. 7: shows a top view of a cell of the lattice structure of the medical device according to the invention according to a further preferred exemplary embodiment, in the expanded state, wherein two webs of a web pair each have a bending site; and

FIG. 8: shows a cross section through a blood vessel with a blood clot and the lattice structure of the medical device according to the invention arranged therein, during use.

The following detailed description of the medical device relates to the lattice structure 10 of the medical device in the production state, i.e. in the completely expanded state of the lattice structure 10, provided that nothing else is specified. The reference point for medical directional specifications, in particular the directional specifications “proximal” and “distal”, is the user of the medical device or the treatment system. Components arranged proximally are therefore closer to the user of the device or the treatment system than distally arranged components.

FIG. 1 illustrates a cut-free, closed cell 15 of a lattice structure 10 of the medical device according to the invention, according to a preferred exemplary embodiment. The cell comprises four webs 11, 12, 13, 14, which are coupled to one another at connection sites 21, 22, 23, 24. In particular, the webs 11, 12, 13, 14 are integrally connected to one another at the connection sites 21, 22, 23, 24. The webs 11, 12, 13, coupled to one another therefore delimit the cell 15. The cell 15 substantially has a diamond-shaped basic shape. Specifically, the connection sites 21, 22, 23, 24 form the corner points of a diamond, with the webs 11, 12, 13, 14 substantially extending along the sidelines of the diamond and connecting the connection sites 21, 22, 23, 24 to one another. Here, the webs 11, 12, 13, 14 do not strictly follow the connection lines between the connection sites 21, 22, 23, 24, but can rather have a shape that deviates from the straight-lined profile of the connection lines. Nevertheless, the basic shape of a diamond remains identifiable. The cell 15 preferably has the diamond-shaped basic shape at least in one state of the lattice structure 10, i.e. in a compressed state or in an expanded state or an intermediate state. In at least one further state of the lattice structure 10, the cell 15 preferably forms a parallelogram-like basic shape. The parallelogram-like basic shape differs from the diamond-shaped basic shape by virtue of the fact that the diagonal connection lines between in each case two connection sites 21, 22, 23, 24 are aligned orthogonal to one another in the diamond shape. In the parallelogram-like basic shape, the diagonal connection lines between two opposing connection sites 21, 22, 23, 24 have an angle in relation to one another which deviates from a right angle. In other words, provision is made in the diamond-shaped basic shape for the connection sites 23, 24, arranged opposite to one another in the longitudinal direction LR of the lattice structure 10, to be arranged in a longitudinal sectional plane LSE of the lattice structure 10 in which the longitudinal axis of the lattice structure 10 also extends. The connection sites 21, 22, 23, 24, arranged opposite to one another in the circumferential direction UR of the lattice structure 10, are arranged in a cross-sectional plane QSE of the lattice structure 10 in the case of the diamond-shaped basic shape, which cross-sectional plane is arranged orthogonally with respect to the longitudinal axis or the longitudinal sectional plane LSE of the lattice structure 10. By contrast, in the parallelogram-like basic shape, the connection sites 23, 24, arranged opposite to one another in the longitudinal direction LR of the lattice structure 10, are offset to one another in the circumferential direction UR such that the diagonal connection line between connection sites 23, 24, arranged opposite to one another in the longitudinal direction, intersects the cross-sectional plane QSE at an angle.

The cell 15 has a first web 11, a second web 12, a third web 13 and a fourth web 14. The first web 11 extends between a first connection site 21 and a third connection site 23. The second web 12 connects the first connection site 21 with a fourth connection site 24. The third web 13 is coupled to the second web 12 by the fourth connection site 24 and to the fourth web 14 by a second connection site 22. The fourth web 14 connects the second connection site 22 to the third connection site 23. The first connection site 21 and the second connection site 22 are arranged opposite to one another in the circumferential direction UR of the lattice structure 10. The third connection site 23 and the fourth connection site 24 are arranged opposite to one another in the longitudinal direction LR of the lattice structure 10.

The first web 11 and the third web 13 are arranged diagonally opposite to one another in the cell 15 and are respectively connected to one another by the second web 12 and the fourth web 14. The first web 11 and the third web 13 together form a first web pair 16. The second web 12 and the fourth web 14 are arranged diagonally opposite to one another in relation to the cell 15 and form a second web pair 17.

The web pairs 16, 17 each have webs 11, 12, 13, 14 with the same design. In particular, the webs 11, 13 of the first web pair 16 substantially have the same shape and the same dimensions, in particular in relation to web width and web thickness. The same applies to the webs of the second web pair, i.e. the second web 12 and the fourth web 14. However, the webs 11, 12, 13, 14 of different web pairs 16, 17 differ from one another in terms of their shape and/or web width. In particular, the webs 11, 13 of the first web pair 16 have such a different shape and/or such different dimensions in relation to the webs 12, 14 of the second web pair 17 that the webs 11, 13 of the first web pair 16 are more deformable than the webs 12, 14 of the second web pair during the transition of the lattice structure 10 from a radially expanded state into a radially compressed state and vice versa. What this achieves is that the third connection site 23 and the fourth connection site 24 move in opposite directions along the circumferential direction UR of the lattice structure 10 during the state change of the lattice structure 10, i.e. they become offset to one another. Particularly during the compression of the lattice structure 10, the third connection site 23 and the fourth connection site 24 or, in general, the connection sites 23, 24 arranged lying opposite to one another in the longitudinal direction LR of the lattice structure 10 are deflected in the opposite direction from the original position in the longitudinal sectional plane LSE in such a way that a distance sets in between the third connection site 23 and the fourth connection site 24 in the circumferential direction UR of the lattice structure 10, as illustrated by the double-headed arrow in FIG. 2.

FIG. 2 shows the cell as per FIG. 1 in the compressed state, wherein it is possible to identify that, as a result of the higher deformability of the webs 11, 13 of the first web pair, there is a deflection of the third connection site 23 and the fourth connection site 24 in such a way that the cell 15 transitions from a diamond-shaped basic shape into a parallelogram-like basic shape during the compression of the lattice structure 10. During the compression of the lattice structure 10, the first connection site 21 and the second connection site 22 approach one another, as symbolized by the block arrows in FIG. 2. The cell 15 is stretched at the same time. This means that the third connection site 23 and the fourth connection site 24 move apart in the longitudinal direction LR of the lattice structure 10. In the process, the third connection site 23 and the fourth connection site 24 also move apart in the circumferential direction UR of the lattice structure 10, and so, substantially, it is possible to speak of a rotation of the cell 15 about a center of rotation arranged within the cell. Since the cell 15 is part of a circumferential segment 20 of the lattice structure 10, which comprises a plurality of cells, more particularly a plurality of cells 15 with the same design, and forms a closed cell ring, an offset of the connection sites 21, 22, arranged opposite to one another in the circumferential direction, i.e. the first connection site 21 and the second connection site 22, is avoided. This leads to the state change of the lattice structure 10, i.e. the compression or the expansion, only having an effect on an offset between the connection sites 23, 24 arranged opposite to one another in the longitudinal direction LR of the lattice structure 10.

In the exemplary embodiment as per FIGS. 1-4, the increased deformability of the webs 11, 13 of the first web pair 16 is achieved by the particular shape of the first web 11 and the third web 13. In particular, the first web 11 and the third web 13 are substantially bent in an S-shape. In other words, the first web 11 and the third web 13 exhibit an S-shaped profile between their respective connection sites 21, 22, 23, to the webs 12, 14 of the second web pair 17. By contrast, the webs 12, 14 of the second web pair 17 extend in a straight line between their respective connection sites 21, 22, 23, 24. Hence, the second web 12 and the fourth web 14 have a lower deformability or a higher rigidity than the first web 11 and the third web 13. During the state change of the lattice structure 10, the first web 11 and the third web 13 therefore deform more strongly than the second web 12 and the fourth web 14. Thus, in general, the webs 11, 13 of the first web pair 16 can bend more or are more flexible than the webs 12, 14 of the second web pair 17. The deformability or bendability and/or flexibility of the first web 11 and the third web 13, i.e. the webs of the first web pair 16 amongst themselves, is substantially equal. The second web 12 and the fourth web 14, i.e. the webs 12, 14 of the second web pair 17 amongst themselves, likewise have the same deformability or bendability and/or flexibility.

The medical device in general has a lattice structure 10 which comprises a multiplicity of cells 15. In particular, the lattice structure 10 comprises circumferential segments 20 which have a plurality of cells 15. The circumferential segments 20 each form a cell ring of cells 15, which extends about the longitudinal axis of the lattice structure 10. The circumferential segments 20 of the lattice structure 10 are connected to one another in the longitudinal direction LR of the lattice structure 10, and so, overall, this forms a closed lattice structure 10. The cells 15 of an individual circumferential segment 20 have the same design. This ensures that the same offset of the connection sites 23, 24, arranged opposite to one another in the longitudinal direction, is set in each individual circumferential segment 20.

In general, the lattice structure 10 can have an integral design. In particular, the lattice structure 10 can be produced integrally from a solid material by cutting out the cell openings. Here, the webs 11, 12, 13, 14 are exposed by stripping away material in the cells 15. The lattice structure 10 is preferably produced by laser cutting or forms a laser-cut lattice structure 10. The lattice structure 10 has a circular cylindrical design, at least in sections. The lattice structure 10 therefore forms a wall plane, which extends in a circular cylindrical shape about the longitudinal axis of the lattice structure 10. In this manner, a lattice structure 10 which is like a tubule, in particular like a stent, is formed, at least in sections.

It is possible to identify from FIG. 3 that several circumferential segments 20 with cells 15 with the same design can form the lattice structure 10. In particular, FIG. 3 shows a section of the lattice structure 10, wherein three circumferential segments 20 are illustrated, which each comprised cells 15, wherein the cells 15 of all three circumferential segments 20 have the same design. In particular, the cells 15 each have two web pairs 16, 17, wherein the first web pair 16 has S-shaped bent webs 11, 13 and the second web pair 17 has webs 12, 14 with a straight design. FIG. 3 shows the expanded state of the lattice structure 10, wherein the cells 15 have a diamond-shaped basic shape. The dashed lines extending vertically in the plane of the drawing on the one hand show the limits of the circumferential segments 20. On the other hand, the dashed lines extending vertically show the position of individual cross-sectional planes QSE, in which connection sites 21, 22, 23, 24 of the webs 11, 12, 13, 14 are arranged in each case. The connection sites 21, 22, 23, 24 move along the cross-sectional planes QSE during the state change of the lattice structure 10, i.e., for example, during the transition from the expanded state into the compressed state. Here, the connection sites 23, 24, arranged opposite to one another in the longitudinal direction, of the individual cells 15 move in opposite directions along the circumferential direction UR of the lattice structure 10. In the lattice structure as per FIG. 3, there is an offset of the connection sites 23, 24, arranged opposite to one another in the longitudinal direction LR of the lattice structure 10, i.e. the third and fourth connection sites 23, 24 of the cells 15, due to the compression. Since the connection sites 23, 24, arranged opposite to one another in the longitudinal direction, of all cells 15 of adjacent circumferential segments 20 become offset to one another, there is, overall, a twist in the lattice structure 10 during the transition from the radially expanded state, as illustrated in FIG. 3, into the radially compressed state, as shown in FIG. 4. It can easily be identified in FIG. 4 that the offset of the connection sites 23, 24, arranged opposite to one another in the longitudinal direction, of the individual cells 15 achieves a rotation of the individual circumferential segments 20 of the lattice structure 10 with respect to one another. Hence, a screw-like twist movement of the lattice structure 10 is brought about simply by the radial expansion or compression of the lattice structure 10.

During use, what the twist of the lattice structure 10, which occurs firstly during the expansion and secondly during the compression as well, achieves is that the webs 11, 12, 13, 14 of the lattice structure 10 for example cut into a blood clot 31 in a screw-like fashion, as illustrated in FIG. 8 in an exemplary fashion. FIG. 8 shows a cross section through a blood vessel 30, in which blood clot 31 is arranged. It furthermore schematically illustrates a cross section through the lattice structure 10, wherein it can be identified that, during the expansion of the lattice structure 10, the webs 11, 12, 13, 14 of the lattice structure 10 cut into the blood clot 31 not only in the radial direction, proceeding from the longitudinal axis of the lattice structure 10, but also engage in the blood clot 31 in the circumferential direction UR of the lattice structure 10. The circumferential direction UR or the movement of the webs 11, 12, 13, 14 directed in the circumferential direction is illustrated by arrows in FIG. 8. Thus, undercuts are formed in the blood clot 31, which undercuts contribute to better adhesion of the blood clot 31 on the lattice structure 10.

Structurally, the twist of the lattice structure 10 is based on the different design of the webs of the first web pair 16 and of the second web pair 17. In the exemplary embodiment as per FIGS. 1-4, the different shape of the first and third web 11, 13 compared to the second and fourth web 12, 14 for example brings about the different deformability of the webs 11, 13 of the first web pair 16 compared to the webs 12, 14 of the second web pair 17 and, as a result, brings about the twist of the lattice structure 10. Alternatively, or in addition thereto, provision can be made for the deformability and/or bendability or flexibility of the individual webs 11, 12, 13, 14 to be set by a variation in the web width or, in general, the web dimensions. Such a variant is implemented in the exemplary embodiment as per FIGS. 5 and 6. FIG. 5 shows a cut-free, closed cell 15, which is delimited by webs 11, 12, 13, 14. Analogously to the exemplary embodiment as per FIGS. 1-4, the webs 11, 12, 13, 14 are connected to one another at connection sites 21, 22, 23, 24. In the expanded state, the cell 15 has a diamond-shaped basic shape.

The webs 11, 12, 13, 14 of the cell 15 as per FIG. 5 substantially have an S-shaped profile between two connection sites 21, 22, 23, 24. Thus, the shape of the webs 11, 12, 13, 14 of the cell 15 is substantially the same. However, the first web 11 and the third web 13, i.e. the webs 11, 13 of the first web pair 16, have a web width which is smaller than the web width of the second web 12 and the fourth web 14, i.e. the first 12, of the second web pair 17. The web width of the first and third web 11, 13, i.e. the webs 11, 13 of the first web pair 16 amongst themselves, is the same. The second web 12 and the fourth web 14, i.e. the webs 12, 14 of the second web pair 17, likewise have the same web width. The webs with reduced web width, in particular the first web 11 and the third web 13, are therefore more deformable than the webs 12, 14 of the second web pair 17. As a result, there is an offset of the third connection site 23 and the fourth connection site 24 in the circumferential direction UR of the lattice structure 10 during the state change of the lattice structure 10, as illustrated in FIG. 6. Hence, the cell 15 twists overall. Since the cell 15 is part of a circumferential segment 20, which is made of cells 15 with the same design, there is, overall, a twist of the lattice structure 10 during a state change, in particular an expansion or compression of the lattice structure 10.

The offset of the connection sites 23, 24, arranged opposite to one another in the longitudinal direction, of the individual cells 15 is the same due to the pair-by-pair arrangement of the webs 11, 12, 13, 14. This means that the third connection site 23 is deflected from a rest position in the circumferential direction of the lattice structure 10 by the same absolute value as the fourth connection site 24 as well. The magnitude of the deflection is therefore the same, wherein, however, the direction of the deflection is different. By way of example, the third connection site 23 can be deflected in the clockwise direction from the rest position during the compression of the lattice structure 10, whereas the fourth connection site 24 is deflected in the counterclockwise direction from the rest position.

A further option of setting the deformability, bendability or flexibility of the webs 11, 12, 13, 14 of the lattice structure 10 differently consists of changing the shape and/or the dimensions of the webs 11, 12, 13, 14, at least in sections. By way of example, it is possible for bending sites 18 to be provided, which increase the deformability of individual webs 11, 12, 13, 14. By way of example, the bending sites 18 can be formed by tapering, wherein the web width of a web 11, 12, 13, 14 is reduced in sections. Alternatively, or in addition thereto, the web thickness of a web 11, 12, 13, 14 can also be reduced in sections at the bending sites 18.

In the exemplary embodiment as per FIG. 7, provision is made for the first web 11 and the third web 13, i.e. the webs 11, 13 of the first web pair 16, to have a bending site 18 each. The bending site 18 forms a section of the respective web 11, 13, in which the web width is reduced compared to the web width of the webs 12, 14 of the second web pair 17. As a result, overall, the first web 11 and the third web 13 have a greater flexibility than the second web 12 and the fourth web 14. In the case of a state change of the lattice structure 10, i.e., for example, during the transition of the lattice structure 10 from the expanded state into the compressed state, the first web 11 and the third web 13 are therefore more deformable than the second web 12 and the fourth web 14. During the state change of the lattice structure 10, the webs 11, 13 of the first web pair 16 therefore deform more strongly than the webs 12, 14 of the second web pair 17. Hence, overall, the cell 15 deforms and changes from a diamond-shaped basic shape in the expanded state, as illustrated in FIG. 7, into a parallelogram-like basic shape, wherein the connection sites 23, 24, arranged opposite to one another in the longitudinal direction, of the cell 15 are offset to one another.

The lattice structure 10 is preferably part of a treatment system, wherein the lattice structure 10 has a proximal axial end which is fixedly, more particularly rotationally fixedly, connected to a distal end of a guide wire. During the use of the treatment system, the proximal end of the lattice structure 10 is therefore held substantially stationary by the guide wire, such that the webs 11, 12, 13, 14 of the lattice structure 10 can cut into a blood clot 31 in a screw-shaped manner when the lattice structure 10 expands. The expansion of the lattice structure 10 is preferably brought about independently. The lattice structure 10 preferably has a self-expanding design. By way of example, the lattice structure 10 comprises shape memory material, in particular a nickel titanium alloy, which brings about the self-expanding properties.

Within the scope of the application, the ratio between the rotation of the individual circumferential segments 20, i.e. the offset between the connection sites 23, 24, arranged opposite to one another in the longitudinal direction, of a circumferential segment 20, and the change in diameter during the expansion or compression of the lattice structure 10 is referred to as degree of rotation. The degree of rotation is determined for each circumferential segment 20. It is possible that the degree of rotation changes or is varied along the lattice structure 10. By way of example, this can be achieved by virtue of the fact that different circumferential segments 20 have a different degree of rotation. The degree of rotation can be set by suitable dimensioning of the individual web pairs. Thus, different circumferential segments can comprise cells 15 with different designs, wherein the cells 15 in one circumferential segment 20 are the same. The different circumferential segments 20 can bring about a change in the degree of rotation along the lattice structure 10. In other words, the dynamics of the rotation can change during an expansion of the lattice structure 10. By way of example, a circumferential segment 20 arranged proximally can rotate more slowly during the expansion of the lattice structure 10 than a circumferential segment 20 arranged more distally. Here, it is also possible that the direction of rotation of individual circumferential segments 20 differs. The direction of rotation of the lattice structure 10 can thus change along the lattice structure 10. By way of example, individual circumferential segments in a proximal region of the lattice structure 10 can rotate in the clockwise direction, whereas circumferential segments in a distal region of the lattice structure 10 rotate in the counterclockwise direction. In an extreme case, provision can be made for a central section of the lattice structure 10 to twist, whereas the respective axial ends of the lattice structure 10 do not carry out a relative movement with respect to one another in the circumferential direction of the lattice structure 10. In any case, the rotation or twist of the lattice structure 10 is already caused solely by the radial expansion or compression.

The treatment system can also comprise more than one lattice structure 10. By way of example, two lattice structures 10 can be superposed such that, during the expansion of the two lattice structures 10, a shear movement is set between the webs of the lattice structures 10. In other words, two or more lattice structures 10 can be arranged within one another.

The device according to the invention and, in particular, the treatment system according to the invention are suitable for different usage purposes. By way of example, blood clots 31 or thrombi can be separated or peeled off a vessel wall with the aid of the device according to the invention. Here, the lattice structure 10 is expanded against the vessel wall up to the stop and, in the process, rotates between the vessel wall and the blood clot 31. Here, the expansion preferably takes place by pushing the lattice structure 10 out of a supply catheter, wherein the supply catheter is held in a stationary manner. The distal end of the lattice structure 10 can be rounded off, particularly for this usage purpose, in order to have an atraumatic effect, i.e. in order to avoid damage to a vessel. Alternatively, the distal end of the lattice structure 10 can have cutting edges, which promote a separation of the blood clot from the vessel wall.

A further field of use of the device according to the invention or of the treatment system consists of treating plaque. By way of example, plaque in blood vessels can be removed layer-by-layer. The torsional movement of the lattice structure 10 in this case has a similar effect to that of a mill, which removes the plaque layer-by-layer. For this usage purpose, the lattice structure 10 preferably has a design with comparatively wide meshing, i.e. it has relatively large cell openings. The lattice structure 10 furthermore comprises comparatively stable webs 11, 12, 13, 14, and so the lattice structure 10 has great radial strength. The expansion of the lattice structure 10 for removing plaque is preferably brought about by virtue of the fact that the lattice structure 10 is held stationary and a catheter enveloping the lattice structure 10 is withdrawn in the proximal direction.

The device according to the invention or the treatment system can furthermore be employed to destroy a blood clot. Here, the lattice structure 10 is not expanded completely to the vessel wall, but rather it is anchored in the blood clot. Provision can be made, particularly for the aforementioned usage purpose, for the medical device to be arranged within a protective basket, in which the blood clot is encapsulated. Such a treatment system, which comprises a protective basket, in which the medical device or the lattice structure 10 is arranged, is not restricted to this usage purpose.

Furthermore, the medical device, particularly in conjunction with the treatment system, can be used in combination with a suction unit. The treatment system specifically comprise a suction unit, which is coupled to the lattice structure 10 or to the cavity or hollow channel spanned by the lattice structure 10. Here, the removal of particles by suction by means of the suction unit can, for example, take place within the lattice structure 10. To this end, provision can advantageously be made for the lattice structure 10 to have a coating such that the negative pressure used up by the suction unit substantially only acts on the blood clot to be removed and a removal of blood from a blood vessel by suction is largely avoided. In particular, the coating can have such a design that the cell openings of the cells 15 are covered in a fluid-tight fashion. Alternatively, the removal by suction can also be brought about by a separate device. Additionally, a basket-like element can be employed, into which the removed blood clot or particles of the removed blood clot are suctioned. The medical device according to the invention or the lattice structure 10 of the device can be expanded into the blood clot in such a way that the blood clot is destroyed. The separated particles of the blood clot can subsequently be removed by means of the separate suction apparatus.

In general, the device according to the invention or the treatment system according to the invention is suitable not only for removing blood clots from blood vessels, but, in general, also for removing different types of concretion from hollow body organs.

The lattice structure 10 can preferably be compressed in such a way that it can be inserted into a supply catheter which has an internal diameter of less than 1.8 mm, in particular of less than 1.4 mm, in particular of less than 1.0 mm, in particular of less than 0.72 mm, in particular of less than 0.05 mm, in particular of less than 0.42 mm.

### List of Reference Signs

10 Lattice structure

11 First web

12 Second web

13 Third web

14 Fourth web

15 Cell

16 First web pair

17 Second web pair

18 Bending site

20 Circumferential segment

21 First connection site

22 Second connection site

23 Third connection site

24 Fourth connection site

30 Blood vessel

31 Blood clot

LSE Longitudinal sectional plane

QSE Cross-sectional plane

LR Longitudinal direction

UR Circumferential direction

1. A medical device with a compressible and expandable, circular cylindrical lattice structure comprising circumferential segments made of closed cells, wherein the cells are each delimited by four webs which are coupled to one another at connection sites and of which two webs, respectively arranged opposite to one another, have the same design and form a web pair, wherein the webs of a first web pair have, at least in sections, a different shape and/or a different web width than the webs of a second web pair in such a way that the webs of the first web pair are more deformable during the transition of the lattice structure from the expanded state into the compressed state than the webs of the second web pair, wherein respectively one web of the first web pair is coupled to one web of the second web pair in such a way that two connection sites arranged opposite to one another in the longitudinal direction LR of the lattice structure are offset in the opposite direction in the circumferential direction UR of the lattice structure during the transition of the lattice structure from the expanded state into the compressed state, wherein all cells of a circumferential segment have the same design such that the whole lattice structure twists, at least in sections, during the transition from the expanded state into the compressed state.

2. The device as claimed in claim 1, wherein each cell comprises four connection sites, which span a diamond-shaped basic shape of the cell in the expanded state of the lattice structure.

3. The device as claimed in claim 1, wherein the webs of the first web pair have a substantially S-shaped embodiment and the webs of the second web pair have a substantially straight embodiment.

4. The device as claimed in claim 1, wherein the webs of the first web pair have a web width which is less than the web width of the webs of the second web pair.

5. The device as claimed in claim 1, wherein the webs of the first web pair each have at least one bending site, at which the web width and/or the web thickness of the respective web is reduced or increased in sections.

6. The device as claimed in claim 1, characterized in that wherein the webs are integrally connected at the connection sites.

7. The device as claimed in claim 1, wherein the circumferential segments each comprise two partial segments, which each have webs arranged in a meandering fashion, wherein every second web of a partial segment has the same design.

8. A treatment system with the medical device as claimed in claim 1 and with a catheter, in which a guide element is arranged in a longitudinally displaceable fashion, wherein the guide element is fixedly, more particularly rotationally fixedly, connected to a proximal end of the lattice structure of the medical device.
